(19) United States
(12) Patent Application Publication
(10) Pub. No.: US 2010/0187108 A1
(43) Pub. Date: Jul. 29, 2010

(54) GAS-COMPONENT MEASUREMENT DEVICE

(76) Inventors: Toru Matsumoto, Minato-ku (JP); Hidehiko Kuroda, Minato-ku (JP); Hideyuki Sato, Minato-ku (JP)

Correspondence Address: SUGHRUE MION, PLLC, 2100 PENNSYLVANIA AVENUE, N.W., SUITE 800, WASHINGTON, DC, 20037, US

(21) Appl. No.: 12/665,460

(22) PCT Filed: Jun. 18, 2008
(86) PCT No.: PCT/JP2008/061090

§ 371 Date: Dec. 18, 2009

(30) Foreign Application Priority Data
Jun. 20, 2007 (JP) 2007-162496

(51) Int. Cl. C12Q 1/00; G01N 27/26

(52) U.S. Cl. 204/403.14; 204/431

(57) ABSTRACT

A gas-component measurement device includes a housing having an suction port that introduces a measurement-targeted gas, and an exhaust port that discharges the measurement-targeted gas, and a water-absorbing member that is disposed in the housing and impregnated with a solvent that dissolves a gas component, and an electrochemical sensor that detects the gas component trapped by the solvent in the water-absorbing member. The exhaust port and suction port are disposed to oppose each other while sandwiching therebetween the electrochemical sensor.

### TECHNICAL FIELD

The present invention relates to a gas-component measurement device that measures or detects a gas component in a measurement target and, more particularly, to a gas-component measurement device that uses an electrochemical sensor.

### BACKGROUND ART

A measurement device that includes an electrochemical sensor is known as the gas-component measurement device that measures or detects (hereinafter referred to as simply “measures”) a specific component in a measurement-targeted gas.

The gas-component measurement device generally includes a reaction chamber or reaction vessel that receives therein a measurement-targeted gas component and allows the electrochemical sensor to respond thereto. The measurement device receives therein an electrochemical sensor and a buffer solution including an electrolyte, wherein the electrochemical sensor is supplied with the buffer solution. The measurement-targeted gas component is dissolved in the buffer solution and thereafter reacted with the electrochemical sensor for a quantitative analysis thereof.

FIG. 6 is a sectional view showing the internal structure of the gas-component measurement device described in Patent Publication-1. In the same figure, a housing 221 of the measurement device includes a gas-sampling room 223 on the top side, and an electrolyte room 224 on the bottom side, wherein both the rooms 223 and 224 are separated from each other by a partition 222. The gas-sampling room 223 is provided with suction and exhaust ports 228, and an inlet port 230 for the electrolyte. The electrolyte room 224 is provided with an outlet port 231 for the electrolyte.

A counter electrode 232 is disposed in a recess of the partition 222, a working electrode 235 is installed within the sampling room 223, and a reference electrode 239 is attached to the outer wall of the electrolyte room 224 while penetrating the same.

The gas-component measurement devices known heretofore include other devices described in Patent Publication-2 and Patent Publication-3. For example, Patent Publication-3 describes a gas detection device wherein a gas detection element and a gas suction unit are installed in separate housings.

Patent Publications-1 to -3 are as follows:

Patent Publication-1—JP-1984-217153A;

Patent Publication-2—JP-1995-77511A; and

Patent Publication-3—JP-1999-153526A

### DISCLOSURE OF THE INVENTION

### Problems to be Solved by the Invention

A structure is desired that allows the measurement-targeted gas component to be steadily received and efficiently supplied to the electrochemical sensor, upon measuring the gas component by using the electrochemical sensor. Use of such a device raises the convenience during the measurement and leads to an improvement in the cleaning, longer lifetime and stability.

However, it is difficult for the gas-component measurement device described in the above Patent Publication-1 to achieve an exact measurement depending on the species of the measurement-targeted gas component. The reason is that the suction port and exhaust port 228 are not arranged in consideration of circulation of the measurement-targeted gas. In this gas-component measurement device, it is needed to dispose a suction fan or increase the size of the suction fan and exhaust fan, whereby it is difficult to reduce the device size.

The problems encountered in the gas-component measurement device described in the above Patent Publication-1 are the common problems in the gas-component measurement devices of Patent Publications-2 and -3.

Thus, it is an object of the present invention to improve the gas-component measurement device described in the above Patent Publications and to thereby provide a gas-component measurement device that facilitates efficient uptake of the gas component in the measurement-target without disposing a suction fan or without the necessity of increasing the size of a suction fan or exhaust fan.

### Means for Solving the Problems

The present invention provides a gas-component measurement device that measures a gas component in a measurement-targeted gas, including: a housing including a suction port that introduces the measurement-targeted gas, and an exhaust port that discharges the measurement-targeted gas; a water-absorbing member disposed in the housing and impregnated with a solvent that dissolves the gas component; and an electrochemical sensor that detects the gas component trapped by the solvent in the water-absorbing member, wherein the suction port and the exhaust port are disposed to oppose each other while sandwiching therebetween the electrochemical sensor.

### EFFECT OF THE INVENTION

According to the gas-component measurement device of the present invention, the gas component in the measurement-targeted gas received from the suction port can be easily discharged from the opposing exhaust port, and the gas component flowing from the suction port via the inside of the housing toward the exhaust port passes through the sensor, thereby facilitating sampling of the gas component.

The above and other objects, features and advantages of the present invention will be more apparent from the following description, referring to the accompanying drawings.

### BRIEF DESCRIPTION OF THE DRAWINGS

FIG. 1 is a schematic longitudinal-sectional view showing the internal structure of a gas-component measurement device according to a first exemplary embodiment of the present invention.

FIG. 2 is a schematic longitudinal-sectional view showing the internal structure of the gas-component measurement device according to the first exemplary embodiment of the present invention.

FIG. 3 is a sectional view of the electrochemical sensor used in the gas-component measurement device of FIGS. 1 and 2.

FIG. 4 is a schematic longitudinal-sectional view showing the internal structure of a gas-component measurement device according to a third exemplary embodiment of the present invention.

FIG. 5 is a schematic longitudinal-sectional view showing the internal structure of a gas-component measurement device according to a fourth exemplary embodiment of the present invention.

FIG. 6 is a longitudinal-sectional view showing the internal structure of a gas-component measurement device described in a patent publication.

### BEST MODE FOR CARRYING OUT THE INVENTION

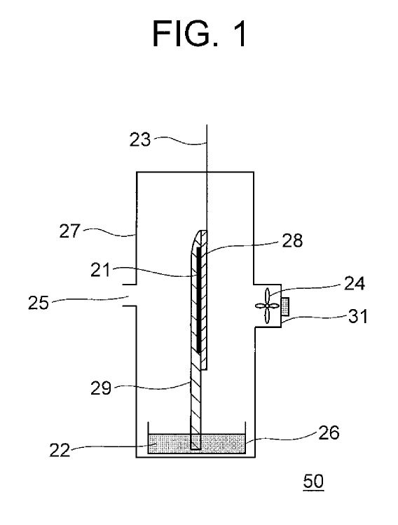

Gas-component measurement devices according to exemplary embodiments of the present invention will be described hereinafter with reference to the drawings. In the drawing, similar constituent elements are shown as designated by similar reference numerals. FIG. 1 is a longitudinal-sectional view of a gas-component measurement device according to a first exemplary embodiment of the present invention.

The gas-component measurement device 50 of FIG. 1 includes a housing 27 having a substantially rectangular solid shape, which receives therein a buffer-solution container 26 that receives therein a buffer solution 22, an electrochemical sensor (referred to simply as sensor hereinafter) 21 that has a function of detecting or measuring a specific component contained in the gas, and a water-absorbing member 29 having an end that is immersed in the buffer solution 22 and another end that is in contact with the electrochemical sensor 21. The sensor 21 is mounted on a substrate 28, which is fixed onto the inside of the housing 27 by a supporting device not illustrated. External wiring 23 configured by a signal line is connected to the sensor 21 via the substrate 28. A suction port 25 that introduces the measurement-targeted gas is formed in a side surface of the housing 27, and an exhaust port 31 that discharges the residual gas component is formed in another side surface that opposes the side surface. An exhaust fan 24 is disposed on the exhaust port 31. Note that the buffer-solution container 26 is not necessarily received within the housing 27.

In the gas-component measurement device 50 of the present exemplary embodiment, the solvent is siphoned by the water-absorbing member 29, and the measurement-targeted gas component is trapped by the solvent in the water-absorbing member 29. Thus, the solvent that contains the measurement-targeted gas component is steadily supplied toward the sensor 21. Accordingly, the gas-component measurement device 50 is quick for response, hardly liable to an influence by the measurement environment, capable of performing a long-time measurement, and thus capable of suitably measuring the specific component in the gas.

The buffer-solution container 26 is disposed on the bottom surface of the housing 27, and accommodates therein the buffer solution 22. The water-absorbing member 29 is made of a strip-shaped porous material, and siphons the buffer solution 22 in the buffer-solution container 26 by a surface tension thereof, to supply the same to the sensor 21. The water-absorbing member 29 has a broad surface that is directed toward the suction port 25, and is exposed to the air flow of the measurement-targeted gas that is introduced from the suction port 25 and flows toward the exhaust port 31. As a result, the detection-targeted gas component is effectively trapped by the water-absorbing member 29, to dissolve into the buffer solution. The gas component thus dissolved is detected by the sensor 21 that is in contact with the rear surface of the water-absorbing member 29. The residual gas component that is not trapped by the water-absorbing member 29 is discharged from the exhaust port 31.

It is sufficient that the housing 27 have a shape and a size that allow the above-described parts to be mounted. For example, plastics is preferably used as the material of the housing 27 in the view point of facilitation of processing, lower cost for the material, and facilitation of handling.

A printed circuit board etc., wherein copper wiring etc. are formed on an insulating substrate, such as made of polyimide resin, are preferably used for the substrate 28 from the viewpoint of reliability and cost. Copper printed wiring formed on the printed circuit board in advance and the terminals of the sensor 21 are connected together, whereby the sensor 21 is connected to the external wiring 23 via the printed wiring.

The buffer solution 22 includes therein a pH buffer and an electrolyte so that biopolymer having catalyst functions, such as an antibody of sensor 21, enzyme and aptamer, functions with stability. For example, a phosphate buffer solution and sodium chloride are suitably used as the buffering agent and electrolyte, respectively, from the view point of facilitation of acquisition and a lower cost. Depending on the measurement target, a minute amount of alcohol may be included therein as an organic solvent. The alcohol is effective for measuring the gas component that is well soluble in an organic solvent.

The buffer-solution container 26 is preferably made of a material that is hardly affected by the buffer solution 22, and plastics that is the same as the material of the housing 27 is preferably used. The water-absorbing member 29 siphons the buffer solution 22 due to a capillarity phenomenon, and has the function of supplying the same to the surface of the sensor 21 at any time. In order to provide such a function, it is preferable to use cotton, paper, etc. having a higher water-absorbing capability, as the water-absorbing member 29. The water-absorbing member 29 may also be made of a polymer material having a higher water-absorbing property, such as urethane. The water-absorbing member 29 and the sensor 21 are preferably adhered closely to each other. In this case, the gas component that dissolves in the water-absorbing member 29 promptly reacts in the sensor 21 for facilitating the detection thereof.

The support member 30 has the function of closely adhering the rear surface of the water-absorbing member 29 onto the surface of the sensor 21, and is directly fixed onto the housing 27, or fixed onto the housing 27 via the buffer-solution container 26. The support member 30 is not limited to any material so long as the material is not affected by the buffer solution 22. Thus, the above-described plastics may be preferably used for the support member 30.

In the present exemplary embodiment, the suction port 25 and the exhaust port 31 are disposed to oppose each other in particular while sandwiching therebetween the electrochemical sensor 21. This arrangement facilitates superior circulation of the gas component within the housing 27, whereby the measurement-targeted gas component is efficiently sampled from the suction port 25 and supplied to the electrochemical sensor 21.

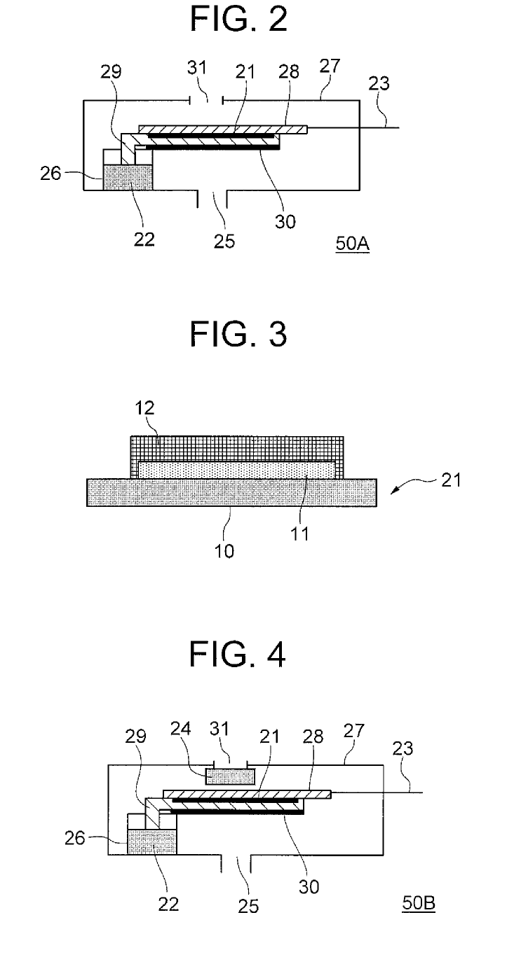

FIG. 2 illustrates a gas-component measurement apparatus according to a second exemplary embodiment of the present invention, similarly to FIG. 1. The gas-component measurement device 50A of the present exemplary embodiment includes a substantially rectangular-solid-shaped housing 27, within which there are provided a buffer-solution container 26 that receives therein a buffer solution 22, a sensor 21 that measures the detection-targeted gas component, a substrate 28 mounting thereon the sensor 21, and a water-absorbing member 29 that supplies the buffer solution 22 toward the sensor 21 from the buffer-solution container 26. The suction port 25 that introduces therethrough the measurement-targeted gas is formed in the bottom surface of the housing 27, and the exhaust port 31 that discharges therethrough the residual gas is formed in the top surface of the housing 27 to oppose the suction port 25 with an intervention of the sensor 21. Signal wiring (external wiring) 23 that extends toward the outside is connected to the substrate 28. One end of the water-absorbing member 29 is immersed in the buffer solution 22, and a tip portion of the water-absorbing member 29 that has a specific length and includes the other end thereof is pressed against the sensor 21 by the support member 30. The substrate 28 that supports the sensor 21 is fixed onto the inside of the housing by a support device not illustrated.

In the present exemplary embodiment, since the suction port 25 is formed in the bottom surface of the housing 27, gas components having a density larger than that of the air are hardly introduced into the housing 27, and other gas components having a density smaller than that of the air are introduced in a larger amount into the housing 27. Therefore, detection of the measurement-targeted gas component having a volatile property is facilitated. Since the tip portion of the water-absorbing member 29 is disposed to oppose the suction port 25 for measuring the gas component at the tip portion, an efficient measurement is achieved. In addition, since the exhaust port 31 is disposed at the location that opposes the suction port 25, circulation of the gas within the housing 27 is facilitated.

The gas-component measurement device 50 of the present exemplary embodiment is suitably used for the case where the detection-targeted gas component has a density smaller than that of the air. In order to sample the gas component with a highest efficiency, the gas-component measurement device 50A is disposed so that the suction port 25 is located at the position where the measurement-targeted gas is generated or is located above the position of an air flow through which the measurement-targeted gas flows. The suction port 25 is not limited to any particular size or shape: however, a round shape is preferable due to facilitation of fabrication. The size of the suction port 25 ranges from several millimeters to several centimeters, for example.

The exhaust port 31 is formed in the top side of the housing 27, and is formed at the position that opposes the suction port 25 with an intervention of the sensor 21 therebetween. Employment of this positional relationship enables a smooth discharge of the gas component that is introduced, and an efficient detection by the sensor 21. The exhaust port 31 is not limited to any size or shape; however, a round shape is preferable due to facilitation of fabrication as in the case of the suction port 25. The exhaust port 31 may be formed using a porous material. It is preferable that the size of the exhaust port 31 range from several millimeters to several centimeters.

FIG. 3 is a sectional view of the electrochemical sensor 21 used in the gas-component measurement device of FIGS. 1 and 2. On an insulating substrate 10, there are formed electrodes 11 including three types of electrodes, wherein an enzyme-immobilizing film, resistor-immobilizing film or aptamer-immobilizing film 12 is formed to cover the electrodes 11. The electrodes 11 include a working electrode, a counter electrode and a reference electrode. Glass or plastics is preferably used as the material of the insulating substrate 10. The working electrode may be of any material so long as the material can detect a current generated at an enzyme reaction or antigen-antibody reaction, and precious metals, such as carbon and platinum, are preferably used. As the working electrode, in particular, precious metals, such as platinum, are preferably used in the case of immobilizing enzyme, whereas carbon is preferably used in the case of immobilizing antibody. This type of sensor using the three-electrode system is superior particularly in the detection sensitivity. Although the above configuration is described in the case of a single sensor, a plurality of sensors may be used for the purpose of detecting the same gas component, or for the purpose of detecting a plurality of gas components.

In the gas-component measurement devices 50 and 50A of the above exemplary embodiments, the measurement-targeted gas including the detection-targeted gas component is introduced into the housing 27 from the suction port 25. A part of the measurement-targeted gas is adsorbed by the water-absorbing member 29 that is in contact with the surface of the sensor 21, and the residual gas that is not adsorbed is discharged from the exhaust port 31. The detection-targeted gas component that is absorbed by the water-absorbing member 29 is promptly dissolved into the buffer solution 22. Since the rate of dissolution of the gas component is higher, and the gas component dissolves in a uniform concentration within the water-absorbing member 29, the response speed of the sensor 21 improves.

FIG. 4 is a longitudinal-sectional view of a gas-component measurement device according to a third exemplary embodiment of the present invention. The gas-component measurement device 50B of the present exemplary embodiment has a configuration similar to that of the gas-component measurement device 50A of the second exemplary embodiment except that an exhaust fan is attached onto the exhaust port 31. In the present exemplary embodiment, description of the constituent elements similar to those in the second exemplary embodiment will be omitted herein.

The gas-component measurement device 50B of the present exemplary embodiment includes the exhaust fan 24 on the inner side of the exhaust port 31. The exhaust fan 24 has the function of discharging gas components from the exhaust port 31 to cause a negative pressure within the housing 27, and receives gas components from the suction port 25. By operating the exhaust fan 24, the measurement-targeted gas is received in the housing 27 from the suction port 25 formed at the position opposing the exhaust fan 24. A part of the gas received in the housing 27 is adsorbed by the tip portion of the water-absorbing member 29 disposed on the front side of the exhaust fan 24, and the residual gas that is not adsorbed is discharged from the exhaust port 31 onto which the exhaust fan 24 is attached.

The detection-targeted gas component absorbed by the tip portion of the water-absorbing member 29 promptly dissolves into the buffer solution 22, to be detected by the sensor 21. In the present exemplary embodiment, provision of the exhaust fan 24 allows the gas component to be sampled efficiently and reliably, received within the housing 27, and supplied to the sensor 21. The response speed of the sensor 21 also improves. The exhaust fan may be positioned on the inner wall of the housing, or may be positioned outside the housing. In the present exemplary embodiment, due to the configuration wherein the exhaust fan is received in the housing 27, all the constituent elements of the gas-component measurement device are settled within the housing, to achieve a simple device structure. The exhaust fan may be a battery-operated fan, or may be operated by an external power source. Another fan may be provided in addition to the exhaust fan, wherein the air flow within the housing may be generated by the another fan.

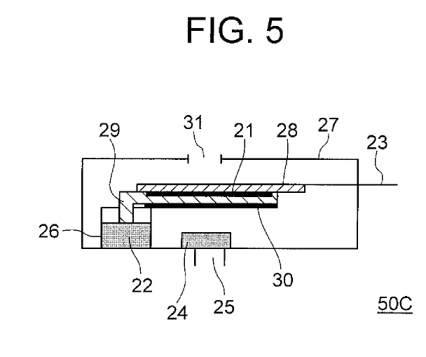

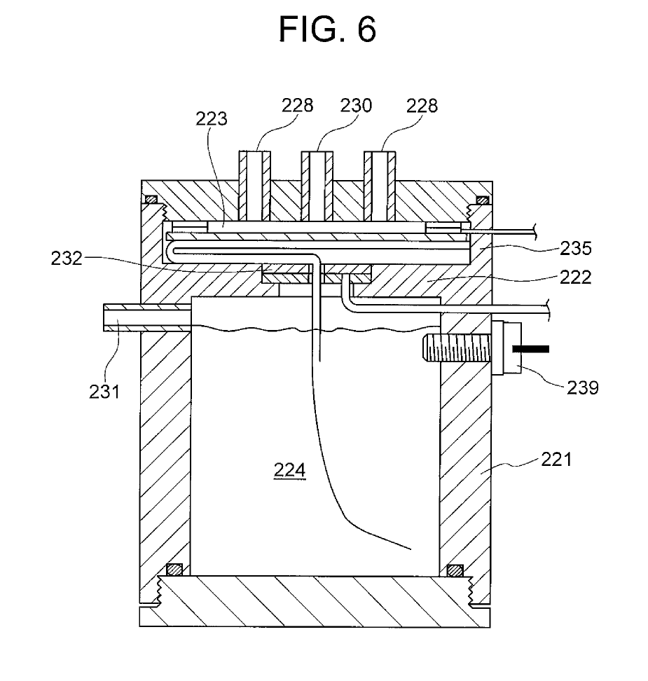

FIG. 5 is a longitudinal sectional view of a gas-component measurement device according to a fourth exemplary embodiment of the present invention. The gas-component measurement device 50C of the present exemplary embodiment is similar to the gas-component measurement device 50A of the second exemplary embodiment except that a suction fan 24 is disposed on the suction port 25.

The gas-component measurement device 50B of the present exemplary embodiment can sample the gas component efficiently and reliably, similarly to the gas-component measurement device of the second exemplary embodiment, due to provision of the suction fan 24 onto the suction port 25. As a result, the sampling efficiency improves. Note that both the suction fan and exhaust fan may be provided. Moreover, another fan may be provided in addition to those.

### EXAMPLES

### Example-1

A gas-component measurement device of example-1 of the present invention was manufactured in accordance with the exemplary embodiment of FIG. 2, for evaluation thereof. The housing was manufactured from polyvinyl chloride having a thickness of 2 mm, to have an inner size which was 50-mm wide, 180-mm long and 50-mm deep. The manufacture used screws as well as encapsulating adhesives. The suction port 25 and exhaust port 31 were disposed to oppose each other while sandwiching therebetween the electrochemical sensor 21. The suction port 25 and exhaust port 31 each had a cylindrical shape of a 25-mm diameter and a 15-mm height, and were made from the same material.

An Eppendorf tube having a 1-ml. (milliliter) volume was fixed onto the bottom surface of the housing 27 as the buffer solution container, which was filled with 0.1-mmol. (millimole) phosphate buffer solution (pH 6.8) containing therein 1-mmol. sodium chloride.

The electrochemical sensor 21 was 10-mm wide, 10-mm long and 0.8-mm thick, and the water-absorbing member 29 made of 1-mm thick polyurethane was adhered onto the surface thereof. An end of the polyurethane was immersed in the Eppendorf tube, and it was confirmed that the phosphate buffer solution covered the surface of the electrochemical sensor.

Manufacture of the electrochemical sensor was performed as described hereinafter. First, a platinum electrode film serving as the working electrode which was 7-mm long and 4-mm wide, a platinum electrode film serving as the counter electrode which was 7-mm long and 1-mm wide, and a silver/silver chloride electrode film serving as the reference electrode were manufactured on a glass substrate by sputtering. The size of glass substrate was 10-mm wide, 10-mm long and 0.8-mm thick.

The silver/silver chloride electrode film was manufactured by sputtering silver and thereafter immersing the same in a ferric chloride solution. Onto the surface of this electrode, alcohol-oxidizing enzyme was immobilized using albumin and glutaraldehyde. Immobilization of the alcohol-oxidizing enzyme was performed using a spin-coat technique. Thereafter, polyurethane was adhered onto the surface as the water-absorbing member. Since the electrochemical sensor is a disposable one, it has a structure facilitating removal thereof.

Subsequently, the external wiring 23 was connected to each electrode of the electrochemical sensor 21, thereby connecting the same to the electrochemical measuring apparatus, “compactstat (registered trademark)”, from Ivium Corporation. Hereinafter, the measurement actually performed and evaluation thereof will be described.

The measurement was such that the current value obtained by applying a constant potential of 0.7V was measured. Evaluation was performed by approaching the measurement device including the housing having a suction port 25 disposed at the bottom thereof toward a beaker that received therein 10-ppm alcohol in a 0.4-ppm hydrogen sulfide ambient, to evaluate the response characteristic until the detection.

As an evaluation result, a sensor response was obtained at a room temperature, about 20° C., when the distance from the beaker was 10 cm. Since the response current was as small as at a nanoampere level, a quantitative evaluation was difficult to achieve; however, it was shown that judgment as to presence or absence of alcohol is possible. Influence by hydrogen sulfide gas was not observed at all.

As a comparative example-1, a measurement device was manufactured having a structure wherein the electrochemical sensor 21 was disposed upward to the contrary, and the gas component was introduced from the opening denoted by 31 in FIG. 2, to be in direct contact with the measuring surface of the electrochemical sensor. In this structure, the gas component is received from the opening 31 formed in the top side of the measurement device, the gas component contacts the electrochemical sensor surface, and the gas component is discharged from the opening 25 formed in the bottom side of the measurement device.

As the result of evaluating the comparative example-1, hydrogen sulfide gas was reacted on the electrode surface immediately after the start of measurement, and the output current value increased along with the reaction, whereby the measurement was impossible due to lack of stability of the base line. It was shown as a result that the gas-component measurement device of the above exemplary embodiment can detect a sample having a higher volatile property.

### Example-2

A gas-component measurement device of example-2 was manufactured in accordance with the exemplary embodiment of FIG. 4, for evaluation thereof. The housing 27 was manufactured similarly to the example-1. The exhaust fan 24 was mounted therein in association with the exhaust port 31. The exhaust fan 24 used herein was a motor fan, F251R, from Copal Electronics Corporation. In the present example, a control board (not shown) for driving the exhaust fan 24 with a size AA battery of 1.5V was newly mounted.

The electrochemical sensor was 4-mm wide, 8-mm long and 0.8-mm thick, and a 0.5-mm-thick polyurethane was adhered onto the surface thereof as the water-absorbing member 29. The tip of the polyurethane was immersed within the Eppendorf tube, and it was confirmed that a phosphate buffer solution covered the electrochemical sensor surface. The working electrode was of a carbon paper which was 2-mm long and 2-mm wide. The counter electrode and reference electrode used herein were similar to those in the example-1.

The above electrodes were adhered onto the surface of a glass substrate, and a trinitrotoluene antibody was immobilized by polyvinyl alcohol. The concrete immobilizing process was such that the glass substrate onto which the carbon paper was adhered was immersed for 30 minutes in a 0.05-mM phosphate buffer solution (pH 7.6) containing therein 0.1-mM sodium chloride in which the trinitrotoluene antibody dissolved, and then immersed in 1% polyvinyl alcohol for 30 minutes.

Subsequently, the glass substrate was immersed in a saturated tryptophan solution, and was dried in a nitrogen ambient for one hour. The carbon paper used was TGP-H-120 supplied from Toray Industries, Inc. Monoclonal antibody supplied from Firmigan Corporation was used as the trinitrotoluene antibody.

Hereinafter, the measurement actually performed and evaluation thereof will be described. The measurement was performed using a rectangular-waveform voltammetry that performs sweeping with a voltage of 0.1V to 1.2V, at a 40-mV amplitude, 20 Hz and a step potential of 15 mV. Note that the sweeping process was iterated by starting again at 0.1V upon reaching 1.2V. The evaluation was performed in a 0.4-ppm hydrogen sulfide gas ambient by operating the exhaust fan 24, with a beaker that received therein a 1000-ppm trinitrotoluene solution dissolved in methanol being disposed at a distance of about 10 cm with respect to the suction port. In this state, the time length needed for obtaining the response current was measured. The suction rate of the gas to the inside of the housing was 0.05 m³/minute.

The gas-component measurement device was gradually approached to the beaker, and it was found that a response that is represented by a current peak is obtained in the vicinity of 0.8V, to thereby detect the trinitrotoluene. Since the response current was as small as at a nanoampere level similarly to the example-1, a quantitative determination was to difficult to achieve; however, it was shown that it is well possible to judge presence or absence of the trinitrotoluene.

As a comparative example-2, another measurement device was manufactured having a structure wherein the surface of the electrochemical sensor was disposed upward, and the gas component from the exhaust port directly contacts the electrochemical sensor. More specifically, the structure is such that the gas component is introduced from the suction port formed on the top side of the measurement device, the gas component contacts the surface of the electrochemical sensor, and the gas component is discharged from the exhaust port formed on the bottom side of the measurement device.

The result was such that the hydrogen sulfide gas reacted on the surface of the electrode at all the potentials from the start of measurement in the gas-component measurement device of comparative example-2, similarly to the comparative example-1, and the output current increased along with the reaction. Thus, the base line was not stabilized, whereby the measurement including the presence or absence of trinitrotoluene was impossible. Accordingly, it was shown that the gas-component measurement device of the present exemplary embodiment is capable of promptly detecting a sample having a higher volatility.

### Example-3

A gas-component measurement device according to an example-3 was manufactured in accordance with the exemplary embodiment of FIG. 5.

In example-3 of the present invention, the suction fan 24 was mounted in the device in association with the suction port 25. The suction fan 24 used herein was an electric fan, F251R, supplied from Copal Electronics Corp. In the present example, a control board (not shown) for driving the suction fan with the size AA battery of 1.5V was newly mounted.

The electrochemical sensor 21 was 4-mm wide, 8-mm long and 0.8-mm thick, and a 0.5-mm-thick polyurethane was adhered onto the surface thereof as the water-absorbing member 29. Thereafter, an end of the polyurethane was immersed in the Eppendorf tube, and it was confirmed that a phosphate buffer solution covered the surface of the electrochemical sensor. The working electrode was of a carbon paper which was 2-mm wide and 2-mm long. The counter electrode and reference electrode used herein were similar to those in example-1.

The above electrodes were adhered onto the surface of a glass substrate, and a trinitrotoluene antibody was immobilized with polyvinyl alcohol. The concrete immobilizing technique was such that the glass substrate onto which the carbon paper was adhered was immersed for 30 minutes in a 0.05-mM phosphate buffer solution (pH 7.6) containing therein 0.1-mm sodium chloride that dissolved the trinitrotoluene antibody, and then immersed in 1-% polyvinyl alcohol for 30 minutes.

Subsequently, the glass substrate was immersed in a saturated tryptophan solution, and was dried under a nitrogen ambient for one hour. TGP-H-120 supplied from Toray Industries, Inc. was used as the carbon paper. A monoclonal antibody supplied from Firmigan Corporation was used as the trinitrotoluene antibody.

Hereinafter, the measurement actually performed and evaluation of the same will be described. Measurement using a rectangular-waveform voltammetry that performs sweeping with a voltage of 0.1V to 1.2V at an amplitude of 40 mV, 20 Hz and a step potential of 15 mV. The sweeping process was iterated again starting at 0.1V upon reaching 1.2V. The evaluation was such that the suction fan was operated with a beaker that received therein a 1000-ppm trinitrotoluene solution dissolved in the methanol being disposed at a distance of 10 cm with respect to the suction port in a 0.4-ppm hydrogen sulfide gas ambient. The time length needed for obtaining the response current was measured. The suction rate was 0.05 m³/minute.

For performing the evaluation, the measurement device was gradually approached to the beaker, and it was found that a response that is represented by a current peak is obtained in the vicinity of 0.8V at a distance of 15 cm to detect the trinitrotoluene. In addition, since the response current was as small as at a nanoampere level similarly to the examples-1 and -2, a quantitative determination was difficult to achieve; however, it was found that it is well sufficient to judge presence or absence of the trinitrotoluene. This result revealed that provision of the suction fan 24 on the suction port 25 enables a higher-sensitive detection. This may be caused by the improvement of the suction rate of the gas component.

As a comparative example-3, a measurement device was manufactured having a structure wherein the electrochemical sensor 21 was disposed upward similarly to comparative example-2, and the gas component from the opening denoted by 31 directly contacts the electrochemical sensor. The evaluation resulted in that the output current increased immediately after the start of measurement at all the potentials along with the reaction of hydrogen sulfide gas with the surface of the electrodes, similarly to comparative examples-1 and -2. Thus, the base line was not stabilized, and measurement including presence or absence of the trinitrotoluene was impossible.

Therefore, it was found that the gas-component measurement device of the above example-3 can promptly detect a sample having a higher volatility.

In the above gas-component measurement devices according to the second to fourth exemplary embodiments of the present invention, the gas component evaporated or sublimated is received from the suction port formed on the bottom side of the housing, trapped by the water-absorbing member, and promptly detected by the sensor. Accordingly, an efficient sampling is achieved. In addition, employment of the configuration wherein the fan is provided in the vicinity of the suction port or exhaust port further improves the measurement sensitivity. On the other hand, the gas-component measurement device hardly inhales a non-volatile substance or non-subliming substance and a substance heavier than the air, or only inhales a small amount thereof if it inhales. Thus, the gas-component measurement device hardly inhales within the housing the hydrogen sulfide gas that is heavier than the air and interferes with the electrochemical sensor. Therefore, the electrochemical sensor is capable of correctly measuring the substance within the gas having the above characteristics. The gas-component measurement device is capable of selectively measuring with a higher sensitivity an explosive component, for example, that is liable to evaporation.

In the second to fourth gas-component measurement devices, it is possible to selectively sample the substance that is likely to evaporate or sublime, whereby the specific component in a gas can be suitably measured, while maintaining a higher measurement accuracy at any time. Since a fan is not operated or since a fan having only a smaller capacity is sufficient, the device can be reduced in size or can measure in a longer-time operation. It is also possible for the gas-component measurement device to measure an explosive substance that is likely to evaporate, such as organic peroxide or low-molecular nitro compound, with a simple process and a sufficient accuracy. Since the suction port is provided downward, contaminants, such as dust and dirt, hardly enter the housing, whereby there is a lower possibility that contaminants adhere onto the sensor. As a result, the gas-component measurement device is capable of performing a longer-time stable measurement.

This application is based upon and claims the benefit of priority from Japanese patent application No. 2007-162496 filed on Jun. 20, 2007, the disclosure of which is incorporated herein in its entirety by reference.

1. A gas-component measurement device that measures a gas component in a measurement-targeted gas, comprising:
a housing including a suction port that introduces the measurement-targeted gas, and an exhaust port that discharges the measurement-targeted gas;
a water-absorbing member disposed in said housing and impregnated with a solvent that dissolves the gas component; and
an electrochemical sensor that detects the gas component trapped by said solvent in said water-absorbing member,
wherein said suction port and said exhaust port are disposed to oppose each other while sandwiching therebetween said electrochemical sensor.

2. The gas-component measurement device according to claim 1, wherein said suction port is disposed on a bottom surface of said housing.

3. The gas-component measurement device according to claim 1, wherein said water-absorbing member siphons said solvent from a container that receives said solvent.

4. The gas-component measurement device according to claim 1, wherein a fan is disposed on at least one of said suction port and said exhaust port.

5. The gas-component measurement device according to claim 4, wherein said fan is disposed within said housing.

6. The gas-component measurement device according to claim 1, wherein said electrochemical sensor includes a biosensor.

7. The gas-component measurement device according to claim 6, wherein said biosensor detects a reaction of a biopolymer that has a catalyst function.

8. The gas-component measurement device according to claim 7, wherein said biopolymer includes at least one of enzyme, antibody, and aptamer.

9. The gas-component measurement device according to claim 6, wherein said electrochemical sensor is a current-detection-type sensor that detects the gas component by a current flowing through a detection electrode.

10. The gas-component measurement device according to claim 9, wherein said electrochemical sensor is a rectangular-waveform voltammetry-type sensor.

11. The gas-component measurement device according to claim 9, wherein said electrochemical sensor includes a reference electrode including a silver/silver chloride electrode.

12. The gas-component measurement device according to claim 1, wherein said solvent includes a substance having a pH-buffering function and an electrolyte.

13. The gas-component measurement device according to claim 1, wherein said solvent includes an organic solvent.

14. The gas-component measurement device according to claim 1, wherein said electrochemical sensor measures an explosive component included in the gas.

15. The gas-component measurement device according to claim 14, wherein said explosive component includes organic peroxide.

16. The gas-component measurement device according to claim 14, wherein said explosive component includes a nitro compound.
